(19) United States
(12) Patent Application Publication
(10) Pub. No.: US 2014/0275399 A1
(43) Pub. Date: Sep. 18, 2014

(54) PREPARATION OF NOVEL FLUOROCOMPOUNDS, METHODS OF PREPARATION AND COMPOSITIONS MADE THEREFROM

(71) Applicant: Henkel US IP LLC, Rocky Hill, CT (US)

(72) Inventors: Tianzhi Zhang, Belle Mead, NJ (US); Matthew P. Burdzy, South Windsor, CT (US); Dingsong Feng, Melrose, MA (US); Yonghui Zhang, Old Saybrook, CT (US)

(21) Appl. No.: 14/245,015

(22) Filed: Apr. 4, 2014

Related U.S. Application Data
(63) Continuation of application No. PCT/US2012/058701, filed on Oct. 4, 2012.
(60) Provisional application No. 61/545,883, filed on Oct. 11, 2011.

(51) Int. Cl. C07C 265/08; C07C 271/14; C08G 18/02; C08F 120/24; C09D 133/16; C08G 77/24; C07F 7/02; C08F 126/02

(52) U.S. Cl. CPC C07C 265/08; C07F 7/025; C07C 271/14; C08F 126/02; C08F 120/24; C09D 133/16; C08G 77/24; C08G 18/02
USPC 524/544; 556/483; 560/355; 560/33; 526/248; 526/245; 525/102; 556/476

(57) ABSTRACT

Novel fluorinated compounds, their method of preparation and use are disclosed, as well as the incorporation of new and old fluorinated compounds in controlled radical polymerization processes to efficiently produce polymer compositions with unique and enhanced properties. Various cure mechanisms and types of end-uses are disclosed.

### FIELD OF THE INVENTION

This invention relates to novel fluorocompounds, their preparation and use, as well as compositions which employ such fluoro compounds. Additionally, this invention relates to the controlled polymerization of compositions containing fluoro compounds, including methods for living radical polymerization of monomers and oligomers with, inter alia, increased conversion, high polydispersity and high functionality.

### BACKGROUND OF THE INVENTION

Fluorinated polymers are known to be useful in many industrial applications due to their unique characteristics, such as high thermo-stability, chemical inertness and low surface energy. Processing of fluorinated polymers can be difficult, however, due to their high melting point and lack of suitable solvents.

Due to these difficulties, as well as certain difficulties in functionalizing fluoro-containing materials, the cost of preparing them is often prohibitively high.

There is a need for new fluorocompounds which can be made using simple and cost effective techniques and which may be used to formulate compositions useful in a variety of areas such as the adhesive, sealant, coating, cleaning, surfactant and water-repellant product areas.

### SUMMARY OF THE INVENTION

The present invention seeks to overcome the processing difficulties associated with fluoropolymers, the manufacture of fluoropolymers, as well as those processing difficulties encountered with compositions made therefrom. The present invention also overcomes the processing difficulties of prior compositions by copolymerizing fluoro-containing monomers with fluoro-free monomers to produce oligomers and polymers with unique physical and processing properties.

The present invention provides a variety of octafluoro compounds and derivatives which can provide enhanced and/or tailored optical, physical, mechanical and chemical properties in the final compositions and products made therefrom.

Additionally, the oligomers and polymers of the present invention can be made efficiently through the use of controlled polymerization methods, such as atom transfer radical polymerization (ATRP), or single electron transfer (SET) polymerization, which provide an efficient and effective means to produce fluoropolymers with reliable and desirable properties on a large scale.

In one aspect of the invention, there is provided novel fluorocompounds and methods of their preparation. These compounds include novel monomers, oligomers and polymers, as well as anionic, cationic and nonionic fluoro-surfactants made therefrom. These new chemical entities are useful in a host of technology and product areas, including, without limitation, the industrial, automotive, electronic and consumer areas. Such products include, without limitation, anaerobic and acrylic adhesives, polyurethane and silicone adhesives, sealants and coatings, as well as cleaners, defoamers and water-repellant products, to name a few. Particularly useful applications include form-in-place (FIP) gasketing applications, cured-in-place (CIP) gasketing applications, injection-molding gasketing applications, photovoltaic applications, fuel cell sealants, Li-ion battery sealant applications, auto-heat exchanger adhesives, and module sealing for various industrial parts.

The fluorocompounds of the present invention may be combined with other reactive and non-reactive components to form compositions with enhanced physical and chemical properties, such as increased temperature and chemical resistance, low coefficient of friction and enhanced electrical properties. In particular, the present compounds and compositions made therefrom have enhanced properties over many current acrylate and silicone products commercially available.

The fluorocompounds may be used to desirably alter the properties of a variety of compositions. They may be used as monomeric additives, they may be grafted onto oligomers or polymers; or they may be grafted onto surfactants to form fluorosurfacts.

In another aspect of the invention, there is provided a composition which includes a compound of the structure I:

[chemical structure]

wherein,

R and R¹ may be the same or different and each may be selected from H, alkyl C₁₋₁₈ substituted or nonsubstituted;

R² may be selected from siloxy, (meth)acryloxy, vinyl ether, epoxy ether, alkyl ether,

[chemical structure]

R³ may be selected from aromatic, aliphatic or cycloaliphatic;

R⁴ may be selected from H, alkyl C₁₋₁₈ substituted or unsubstituted,

[chemical structure]

R⁵ may be selected from an aliphatic or aromatic group which may be substituted or unsubstituted and which may include one or more unsaturated groups;

R⁶ may be selected from a substituted quarternary amine or a metal cation (M+);

R⁷ may be selected from H, alkyl C₁₋₁₈ substituted or unsubstituted, NR³R⁴, OR⁸ or F;

R⁸ may be selected from alkyl C₁₋₂₀ substituted or unsubstituted;

n is 1-4.; and [symbol] indicates the point of attachment to the structure.

In yet another aspect of the invention, there is provided a reaction product of:

i) A composition which includes a compound of the structure I:

[chemical structure]

wherein,

R and R¹ may be the same or different and each may be selected from H, alkyl C₁₋₁₈ substituted or nonsubstituted;

R² may be selected from siloxy, (meth)acryloxy, vinyl ether, epoxy ether, alkyl ether,

[chemical structure]

R³ may be selected from aromatic, aliphatic or cycloaliphatic;

R⁴ may be selected from H, alkyl C₁₋₁₈ substituted or unsubstituted,

[chemical structure]

R⁵ may be selected from an aliphatic or aromatic group which may be substituted or unsubstituted and which may include one or more unsaturated groups;

R⁶ may be selected from a substituted quarternary amine or a metal cation (M+);

R⁷ may be selected from H, alkyl C₁₋₁₈ substituted or unsubstituted, NR³R⁴, OR⁸ or F;

R⁸ may be selected from alkyl C₁₋₂₀ substituted or unsubstituted; and

n is 1-4; [symbol] indicates the point of attachment to the structure; and

ii) a free radical initiator.

In another aspect of the invention, there is provided a method of forming a fluorinated moisture curing silane which includes:

i) mixing an organo silane compound and an alkali or alkaline earth metal oxide in a reaction vessel under heat; and

ii) further combining the resultant mixture with a fluorinated alkanol to form a fluorinated moisture curing silane.

A particularly desirable silane compound is tetramethoxysilane, although other alkoxysilanes may be used. A particularly desirable alkaline earth metal oxide is sodium methoxide; a particularly desirable fluorinated alkanol is 2,2,3,3,4,4,5,5-octylfluoropentanol, although others may be used as later described herein.

In another aspect of the invention there is provided a method of forming a fluorinated curable composition through controlled polymerization comprising:

i.) combining the composition of claim 1 with a free radical initiator, a ligand capable of coordinating with a metal catalyst and a metal catalyst in a reaction vessel;

ii) permitting the reaction to proceed at a suitable temperature and for a suitable time until the desired degree of polymerization is reached, wherein a fluorinated polymerizable compound is produced.

In another aspect of the invention there is provided an adhesive, or sealant or coating composition comprising:

(i) one or more compounds of claim 1;

(ii) one or more reactive components selected from the group consisting of monomers, polymers, oligomers, reactive diluents and combinations thereof; and

(iii) a cure system.

In another aspect of the invention, there is provided a composition which includes a compound which includes in its structure the segment:

[chemical structure]

wherein R₁₀ and R₁₁ may be the same or different and may be independently selected from H and Me; R¹² may be selected from hydrophobic monomers, hydrophilic monomers, aromatic monomers, aliphatic monomers and combinations thereof. Desirably, R¹² is a substituted or unsubstituted C₁₋₂₀ monomer and more desirable than alkyl group C₁₋₂₀. Even more desirably, R¹² is an unsubstituted C₁₋₄ alkly group, eg. —CH₂CH₂CH₂CH₃. R¹² may be substituted with functional groups such as those described herein; and
wherein X represents mole % from 0.001-100 and y represents mole % from 0-99.999.

In another aspect of the invention, there is provided a controlled radical polymerization process which includes:

(i) providing a compound of the structure VI:

[chemical structure]

wherein,

R and R¹ may be the same or different and each may be selected from H, alkyl C₁₋₁₈ substituted or nonsubstituted;

R² may be selected from siloxy, (meth)acryloxy, vinyl ether, epoxy ether, alkyl ether, cyanoacrylate, cyanoacetate.

[chemical structure]

R³ may be selected from aromatic, aliphatic or cycloaliphatic;

R⁴ may be selected from H, alkyl C₁₋₁₈ substituted or unsubstituted,

[chemical structure]

R⁵ may be selected from an aliphatic or aromatic group which may be substituted or unsubstituted and which may include one or more unsaturated groups;

R⁶ may be selected from a substituted quarternary amine or a metal cation (M+);

R⁷ may be selected from H, alkyl C₁₋₁₈ substituted or unsubstituted, NR³R⁴, OR⁸ or F;

R⁸ may be selected from alkyl C₁₋₂₀ substituted or unsubstituted;

n is 1-4; [symbol] indicates the point of attachment to the structure; and R⁹ may be selected from H, alkyl C₁₋₈ substituted or unsubstituted, NR³R⁴, OR⁵ or F;

(ii) combining the compound with a free radical initiator and a chain transfer agent to form a reaction mixture; and

(iii) reacting the resulting mixture at a sufficient temperature and time to form a polymer.

In another aspect of the invention, there is provided a controlled polymerization process which includes:

(i) providing a compound of the structure:

[chemical structure]

wherein,

R and R¹ may be the same or different and each may be selected from H, alkyl C₁₋₁₈ substituted or nonsubstituted;

R² may be selected from siloxy, (meth)acryloxy, vinyl ether, epoxy ether, alkyl ether, cyanoacrylate, cyanoacetate.

[chemical structure]

R³ may be selected from aromatic, aliphatic or cycloaliphatic;

R⁴ may be selected from H, alkyl C₁₋₁₈ substituted or unsubstituted,

[chemical structure]

R⁵ may be selected from an aliphatic or aromatic group which may be substituted or unsubstituted and which may include one or more unsaturated groups;

R⁶ may be selected from a substituted quarternary amine or a metal cation (M+);

R⁷ may be selected from H, alkyl C₁₋₁₈ substituted or unsubstituted, NR³R⁴, OR⁸ or F;

R⁸ may be selected from alkyl C₁₋₂₀ substituted or unsubstituted; R⁹ may be selected from H, alkyl C₁₋₁₈ substituted or unsubstituted, NR³R⁴, OR⁵ or F;

n is 1-4; [symbol] and indicates the point of attachment to the structure.

(ii) combining the compound with an organometallic compound or a hydride of Group IV-VIII transition metals to form a reaction mixture;

(iii) reacting a mixture at a sufficient temperature and time to form a polymer.

In another aspect of the invention, there is provided a controlled polymerization process which includes:

(i) providing a compound of the structure:

[chemical structure]

wherein,

R and R¹ may be the same or different and each may be selected from H, alkyl C₁₋₁₈ substituted or nonsubstituted;

R² may be selected from siloxy, (meth)acryloxy, vinyl ether, epoxy ether, alkyl ether, cyanoacrylate, cyanoacetate.

[chemical structure]

R³ may be selected from aromatic, aliphatic or cycloaliphatic;

R⁴ may be selected from H, alkyl C₁₋₁₈ substituted or unsubstituted,

[chemical structure]

R⁵ may be selected from an aliphatic or aromatic group which may be substituted or unsubstituted and which may include one or more unsaturated groups;

R⁶ may be selected from a substituted quarternary amine or a metal cation (M+);

R⁷ may be selected from H, alkyl C₁₋₁₈ substituted or unsubstituted, NR³R⁴, OR⁸ or F;

R⁸ may be selected from alkyl C₁₋₂₀ substituted or unsubstituted; and R⁹ may be selected from H, alkyl C₁₋₁₈ substituted or unsubstituted, NR³R⁴, OR or F;

n is 1-4; [symbol] indicates the point of attachment to the structure.

(ii) combining the compound with a nitroxide and a free radical initiator.

In another aspect of the invention, there is provided a reactive composition comprising a polymeric or oligomeric backbone and a fluorocompound grafted onto said backbone, said fluorocompound graft including a functionalized octofluoropentyl group.

In another aspect of the invention, there is provided:

A method of forming a fluorinated reactive urethane which includes:

forming a reaction mixture of a diisocyanate compound and an octafluoro alkanol in a reaction vessel at temperatures of less than room temperature; and

adding to the reaction mixture a catalyst and permitting the mixture to warm to room temperature to form the reactive fluorinated urethane.

In another aspect of the invention, the Octafluoro derivatives (OFDs) of the present invention may be in the form of a monomer (FM), an oligomer (FO) or a polymer (FP). Additionally, the OFDs may be grafted onto a polymer (FPG).

For example, when the OFD is a FM, it may be polymerized with itself or another monomer, oligomer or polymer. For example, one FM of the present invention may be polymerized with another monomer (including a non-FM or a different FM of the invention) or the FM may be added to a composition to enhance and/or tailor the properties of a composition.

In another embodiment of the invention, an FO or FP of the present invention may be copolymerized with itself or another polymerizable component in a composition. The FP may be functionalized or non-functionalized. For example, a FO may be added to a monomer composition to lower the surface energy of the composition.

In another aspect of the invention, the OFDs of the present invention may be grafted onto a polymer backbone. A formulation of such a grafted fluoropolymer can thus be provided having enhanced, modified and/or tailored properties.

In yet another aspect of the invention, the OFDs of the present invention may include a surfactant moiety and be formulated into compositions to provide or enhance surfactant properties.

### BRIEF DESCRIPTION OF THE DRAWINGS

FIG. 1 shows the F NMR and H NMR Spectra used to characterize the compound synthesized in Example 1.

FIG. 2 shows the F NMR and H NMR Spectra used to characterize the compound synthesized in Example 2.

FIG. 3 shows the F NMR and H NMR Spectra used to characterize the compound synthesized in Example 3.

FIG. 4 shows the F NMR and H NMR Spectra used to characterize the compound synthesized in Example 4.

FIG. 4A shows the F NMR and the H NMR Spectra used to characterize mono 1-α-2,2,3,3,4,4,5,5-octafluoropentyl urethanyl-α,α-dimethyl methyl,3-isopropenyl-benzene.

FIG. 5 shows a flowchart outlining a useful controlled free-radical polymerization process.

FIG. 6 depicts a proposed SET mechanism useful in the present invention.

FIG. 7 depicts a proposed ATRP mechanism useful in the present invention.

FIG. 8 depicts an Instron apparatus used to test peel strength.

### DETAILED DESCRIPTION OF THE PRESENT INVENTION

For purposes of this invention, the term fluorocompound is meant to include fluoromonomers (FMs) of any structures described herein, as well as fluoro-oligomers (FOs) and fluoropolymers (FPs) made therefrom. Collectively, the FOs, FMs and FPs may be referred to as OFDs (octafluoro derivatives). For purposes of this invention, the term “(meth)acrylate” or “(meth)acryloxy” will include both the methacrylate and acrylate or methacryloxy and acryloxy, respectively.

As used herein for each of the various embodiments, the following definitions apply:

The term “alkyl” is meant to mean straight or branched saturated hydrocarbon groups;

The term “substituted” means substituted with lower alkyl (C₁₋₄), aryl, alkaryl, alkoxy(C₁₋₄), halo; additionally the term may also include a hetero atom such as O or N interrupting the C₁₋₁₈ alkyl chain.

The terms “aromatic” or “aryl” means cyclic conjugated hydrocarbon structures (C₁₋₁₂) which may optionally be substituted as the term “substituted” is defined herein;

The terms “halogen,” “halo” or “hal” when used alone or as part of another group mean chlorine, fluorine, bromine or iodine;

The term “aliphatic” means saturated or unsaturated, straight, branched or cyclic hydrocarbon groups;

The term “oligomer” means a defined, small number of repeating monomer units such as 10-25,000 units, and desirably 10-100 units which have been polymerized to form a molecule, and is a subset of the term polymer; the term “polymer” any polymerized product greater in chain length and molecular weight than the oligomer, i.e. or degrees of polymerization greater than 25,000.

Novel fluoro compounds which have been found to be particularly useful include those represented by Formula I:

[chemical structure]

wherein,

R and R¹ may be the same or different and each may be selected from H, alkyl C₁₋₁₈ substituted or nonsubstituted;

R² may be selected from siloxy, (meth)acryloxy, vinyl ether, epoxy ether, alkyl ether,

[chemical structure]

R³ may be selected from aromatic, aliphatic or cycloaliphatic;

R⁴ may be selected from H, alkyl C₁₋₁₈ substituted or unsubstituted,

[chemical structure]

R⁵ may be selected from an aliphatic or aromatic group which may be substituted or unsubstituted and which may include one or more unsaturated groups;

R⁶ may be selected from a substituted quarternary amine or a metal cation (M+);

R⁷ may be selected from H, alkyl C₁₋₁₈ substituted or unsubstituted, NR³R⁴, OR⁸ or F;

R⁸ may be selected from alkyl C₁₋₂₀ substituted or unsubstituted; and

n is 1-4., and [symbol] indicates the point of attachment to the structure.

Among those found to be particularly useful are set forth in structures II-Vb (described below):

[chemical structure]

wherein each occurrence of R⁹ may be the same or different and may be selected from the group consisting of H, alkyl C₁₋₂₀ and combinations thereof.

[chemical structure]

wherein R³ may be selected from aromatic, aliphatic or cycloaliphatic.

[chemical structure]

In addition to the novel fluorocompounds and their uses and methods of manufacture, the present invention also provides novel methods of using a variety of known fluorocompounds in polymerizable compositions and particularly in compositions made using controlled polymerization reactions such as atom transfer radical polymerization (ATRP), single electron transfer living radical polymerization (SET-LRP) and other controlled radical polymerization methods, as discussed further herein.

Among the fluoromonomers found to be particularly useful are the octafluoromonomers. Table I lists octafluorocompounds which exemplify those found to be particularly useful in the present invention. These and other octafluorocompounds may be used alone or in combination with other reactive components in polymer compositions to make polymerizable materials with tailored properties.

TABLE 1

| | Structure (OFP = |
|---|---|
| Chemical Name | OCH₂(CF₂)₄—H |
| 2,2,3,3,4,4,5,5- Ocafluoropentyl methacrylate | [chemical structure] |
| 2,2,3,3,4,4,5,5- Octafuoropentyl acrylate | [chemical structure] |
| 1,1,2,2,3,3,4,4-Octafluoro-5-(2-propen-1-yloxy)- pntane | [chemical structure] |
| Glycidyl_2,2,3,3,4,4,5,5- Octafluoropentyl ether | [chemical structure] |
| 2,2,3,3,4,4,5,5- Octafluropentoxymethoxysilane mixtures | [chemical structure] |
| 2,2,3,3,4,4,5,5- Octaflurovaleric Acid | [chemical structure] |
| Sodium 2,2,3,3,4,4,5,5- Octafluoro-Pentanoate | [chemical structure] |
| Pantanoic acid,2,2,3,3,4,4,5,5-Octafluoro,-, potassium salt (1:1) | [chemical structure] |
| Ammonium,2,2,3,3,4,4,5,5- Octaflurovalerate | [chemical structure] |
| 1-Pentanol,2,2,3,3,4,4,5,5-octafluoro-,1- (hydrogen sulfate) | [chemical structure] |
| 1-Pentanol,2,2,3,3,4,4,5,5-octafluoro-,1- (hydrogen sulfate),sodium salt (1:1) | [chemical structure] |
| 1-Pentanol,2,2,3,3,4,4,5,5-octafluoro-,1- (hydrogen sulfate),potassium salt (1:1) | [chemical structure] |
| Acetic acid,2-[(2,2,3,3,4,4,5,5- octafluoropentyl)oxy)]- | [chemical structure] |
| 2-Butenedioic acid (2Z)-, mono(2,2,3,3,4,4,5,5-octafluoropentyl)ester | [chemical structure] |
| 2-Butenedioic acid, mono(2,2,3,3,4,4,5,5- octafluoropentyl)ester | [chemical structure] |
| 1,1,2,2,3,3,4,4-octafluro-5-(vinyloxy)pentane | [chemical structure] |

### Polymerizable Compositions

One particularly useful set of fluorinated compounds for incorporation into polymerizable compositions includes without limitation one or more of the compounds from the various structures disclosed herein. Of particular usefulness are octafluorocompounds, which can be incorporated directly as monomers into polymerizable compositions, or chain-extended into oligomers and then incorporated into polymerizable compositions. Additionally, or alternatively, these fluorinated compounds may be grafted onto other compounds such as oligomers or polymer backbones and formulated into polymerizable compositions. Various additional monomers and reactive components may be added into polymerizable compositions made from the fluoromonomers (FMs) and fluoroligomers (FOs) of the present invention.

The octafluoro-derivatives (OFDs, i.e. monomers, oligomers and polymers) of the present invention may be copolymerized with themselves or with other monomers, oligomers and polymers. For example, an FO of the present invention may be added to an FM of the present invention, or another monomer to provide a new composition with enhanced and/or tailored properties. Additionally, the OFDs of the present invention may be added to compositions which, when polymerized, are designed to form separate domains, such as in an interpenetrating network.

### Chain Extension

The fluoro-monomers of the present invention can also be chain-lengthened by polymerization to yield oligomer (FOs) or polymer structures (FPs). For example, the following schematic depicts such chain lengthening in the novel compound 2,2,3,3,4,4,5,5-octafluoropentyl methacrylate.

[chemical structure]

As mentioned, oligomers (FOs) formed from the fluoromonomers may then be further added to other polymerizable compositions to modify the properties of the final cured product.

Additionally, as would be understood from the description of the various fluorocompound structures described herein, the fluoromonomers and fluoroligomers may be functionalized with a wide variety of groups to provide reaction sites for cross-linking, chain extension, or other reactions, such as the addition of other chemical moieties or groups. As further described herein, the selection of functional groups will depend on the desired function and end properties, including altering or enhancing physical and/or chemical properties in the final cured product. Additionally, such functional groups may serve to allow for various cure mechanisms including room temperature cure, heat cure, photoradiation, e.g. UV cure or visible light cure, moisture cure and combinations thereof.

Suitable functional groups for functionalizing the FMs, FOs, and FPs of the present invention include, without limitation, hydroxy, siloxy, epoxy, cyano, halo, isocyanate, amino, aryloxy, aryalkoxy, oxime, (meth)acryloxy, aceto, cyanoacrylate, cyanoacetate, alkyl ether, epoxyether and vinyl ether. In one embodiment, these groups may be added to the fluoromonomer via reaction with compounds containing these functionalities.

The fluorocompounds of the present invention may be incorporated into curable compositions which include a variety of different monomers, oligomers, polymers and reactive diluents. Fluoromonomers may be also grafted onto other compounds, such as other monomers or oligomers and incorporated into curable compositions. These curable compositions may further include cross-linking agents, cure systems, including initiators, accelerators and stabilizing systems, fillers, coloring agents, plasticizing agents, emulsifiers, and other useful components desired or necessary for the chosen cure system.

A variety of different types of polymerizable compositions may be made using the fluoromonomers (FMs), fluoro-oligomers (FOs) and fluoropolymers (FPs) of the present invention. In some embodiments, (meth)acrylate-based monomers may be used in conjunction with FMs, FOs or FPs to form free radical curing adhesives, sealants or coatings. Such compositions may include free radical initiators, such as, but not limited to, those described herein. These compositions may be room temperature cured, heat cured or photoradiation cured, such as by UV or visible light.

In some embodiments, more than one type of cure mechanism may be used. For example, various functional groups may be present as described herein. In the case where an acrylate or vinyl group is present in addition to a siloxy or alkoxy group, free-radical cure and moisture cure would also be available as cure mechanisms. Appropriate free-radical initiators, such as peroxy or perester compounds, and moisture cure catalysts, such as organotins or organotitanates may be used in such compositions. Alternatively, the same composition may be heat cured by selection of a heat cure catalyst, such as a platinum hydrosilylation catalyst.

Various polymeric backbones may be used in constructing polymers which either have the FM directly grafted onto the backbone, or added as a component to the composition. Desirably, the FM or FO is functionalized with one or more of the functional groups described herein, such that it reacts with other polymerizable components to modify their properties in a desirable manner. For example, polyesters, polyolefins, poly(meth)acrylates, polyurethanes, polyurethaneureas, and various combinations and copolymers of these polymers are examples of useful polymer systems which may be modified using the present invention.

In some embodiments, elastomeric compositions such as silicone or polyurethane compositions may be formulated using the FMs, FOs or FPs of the present invention. Such compositions may be useful as adhesives, sealants or coatings. Particularly useful applications include gasketing, such as form-in place (FIP) gasketing, cure-in-place (CIP) gasketing, injection molding applications and photovoltaic applications, to name a few.

In some embodiments, structural (meth)acrylic compositions may be formulated using the FMs, FOs or FPs of the present invention.

Hybrid systems, such as polyurethacrylate, silicone-acrylate and epoxyacrylate, to name a few non-limiting examples, are contemplated as part of this invention. The present invention provides the flexibility of choosing the various reactive components and their respective cure systems to tailor final products and their properties.

As can be appreciated, the compositions of the present invention may take any of the various combinations of components described herein, each of them incorporating one or more of a FM, FO or FP of the present invention.

### Additional Monomer Components

Suitable additional monomers for incorporating into the compositions of the present invention include, without limitation, acrylates, halogenated acrylates, methacrylates, halogen-substituted alkenes, acrylamides, methacrylamides, vinyl sulfones, vinyl ketones, vinyl sulfoxides, vinyl aldehydes, vinyl nitriles, styrenes, and any other activated and nonactivated monomers containing electron withdrawing substituents. These monomers may be substituted. In some embodiments, the monomers optionally contain functional groups that assist in the disproportionation of the metal catalyst into other oxidation states. Functional groups may include without limitation, amide, sulfoxide, carbamate, or onium. Halogen substituted alkenes include vinyl chloride, vinylidene chloride, vinyl fluoride, vinylidene fluoride, trifluoroethylene, trifluorochloroethylene, or tetrafluoroethylene, hexafluorpropylene and fluorinated vinyl esters. Combinations of the monomers may be used. Blends of monomers may be polymerized using the embodiments of the present invention. The monomers may be blended in the reaction vessel. As an example, blends of acrylate monomers may be used with the methods of the present invention, as certain acrylates will exhibit similar reactivities, thus the end product may have a greater predictability. Blends of the final polymer product, as a two co-polymer blend, a two homopolymer blend, and a combination of at least one co-polymer and at least one homopolymer may be blended as may be desired. Further, blended polymers can be made as final products. Blended polymer products may be preferred to others because a blended copolymer may provide and promote good oil resistance in gasket applications. Specifically, the additional monomer may be one or more of for example, alkyl (meth)acrylates; alkoxyalkyl (meth)acrylates; (meth)acrylonitrile; vinylidine chloride; styrenic monomers; alkyl and alkoxyalkyl fumarates and maleates and their half-esters, cinnamates; and acrylamides; N-alkyl and aryl maleimides (meth)acrylic acids; fumaric acids, maleic acid; cinnamic acid; and combinations thereof. More specifically, the monomers used to create polymers with the embodiments of the present invention are not limited to any particular species but includes various monomers, for example: (meth)acrylic acid monomers such as (meth)acrylic acid, methyl(meth)acrylate, ethyl (meth)acrylate, n-propyl (meth)acrylate, isopropyl (meth)acrylate, n-butyl (meth)acrylate, isobutyl (meth)acrylate, tert-butyl (meth)acrylate, n-pentyl (meth)acrylate, n-hexyl (meth)acrylate, cyclohexyl (meth)acrylate, n-heptyl (meth)acrylate, n-octyl (meth)acrylate, 2-ethylhexyl (meth)acrylate, nonyl (meth)acrylate, decyl (meth)acrylate, dodecyl (meth)acrylate, phenyl (meth)acrylate, toluoyl (meth)acrylate, benzyl (meth)acrylate, 2-methoxyethyl (meth)acrylate, 3-methoxybutyl (meth)acrylate, 2-hydroxyethyl (meth)acrylate, 2-hydroxypropyl (meth)acrylate, stearyl (meth)acrylate, glycidyl (meth)acrylate, 2-aminoethyl (meth)acrylate, -(methacryloyloxypropyl)trimethoxysilane, (meth)acrylic acid-ethylene oxide adducts, trifluoromethylmethyl (meth)acrylate, 2-trifluoromethylethyl (meth)acrylate, 2-perfluoroethylethyl (meth)acrylate, 2-perfluoroethyl-2-perfluorobutylethyl (meth)acrylate, 2-perfluoroethyl (meth)acrylate, perfluoromethyl (meth)acrylate, diperfluoromethylmethyl (meth)acrylate, 2-perfluoromethyl-2-perfluoroethylethyl (meth)acrylate, 2-perfluorohexylethyl (meth)acrylate, 2-perfluorodecylethyl (meth)acrylate and 2-perfluorohexadecylethyl (meth)acrylate; styrenic monomers such as styrene, vinyltoluene, alpha-methylstyrene, chlorostyrene, styrenesulfonic acid and salts thereof; fluorine-containing vinyl monomers such as perfluoroethylene, perfluoropropylene and vinylidene fluoride; silicon-containing vinyl monomers such as vinyltrimethoxysilane and vinyltriethoxysilane; maleic anhydride, maleic acid, maleic acid monoalkyl esters and dialkyl esters; fumaric acid, fumaric acid monoalkyl esters and dialkyl esters; maleimide monomers such as maleimide, methylmaleimide, ethylmaleimide, propylmalcimide, butylmaleimide, hexylmaleimide, octylmaleimide, dodecylmaleimide, stearylmaleimide, phenylmaleimide and cyclohexylmaleimide; nitrile-containing vinyl monomers such as acrylonitrile and methacrylonitrile; amido-containing vinyl monomers such as acrylamide and methacrylamide; vinyl esters such as vinyl acetate, vinyl propionate, vinyl pivalate, vinyl benzoate and vinyl cinnamate; alkenes such as ethylene and propylene; conjugated dienes such as butadiene and isoprene; vinyl compounds such as vinyl halides, such as vinyl chloride, vinylidenehalide, allylhalide, allyl alcohol, etc. The aforementioned monomers may be used singly, sequentially, or in combination. From the desirability of physical properties of products, one or more classes of monomer may be preferred.

Among the other useful reactants for incorporation into the inventive compositions include mono, di- and triisocyanates or polymeric-type isocyanates. Di- and triisocyanates are particularly useful. These reactants can be used for linking polyfunctional compounds onto the inventive fluorinated monomers, oligomers or polymers as described herein. Non-limiting examples include ethylene diisocyanate, 1,2-diisocyanatopropane, 1,3-diisocyanatopropane, 1,6-diisocyanatohexane, 1,4-diisocyanatobenzene, para-, meta- and ortho-diisocyanatobenzene, bis(4-isocyanatocyclohexyl) methane, bis(4-isocyanatophenyl) methane (MDI), toluene diisocyanate (TDI) e.g. 2,4-TDI,2,6-TDT, 3,3′-dichloro-4,4″-diisocyanatobiphenyl, tris(4-isocyanatophenyl) methane, 1,5-diisocyanatonapthalene, hydrogenated toluene diisocyanate, 1-isocyanatomethyl-5-isocyanato-1,3,3-trimethylcyclohexane, 1,3,5-tris(6t-isocyanatohexyl) biuret and combinations of any of these.

The reaction of the di- or triisocyanate with alcohol or amine groups on the fluorinated monomers or oligomers of the invention permit formation of urethane or urea groups, as well as provide additional isocyanate functionality for further reaction.

Free radical initiators useful in formulating polymerizable compositions containing FMs, FOs or FPs of present invention include, without limitation, peroxy and perester compounds such as benzoyl peroxide, 2,4-dichlorobenzoyl peroxide, t-butyl perbenzoate, cumene hydroperoxide (CHP), di-t-butyl peroxide and dicumyl peroxide, 2,5-bis(t-butylperoxy) 2,5-dimethylhexane. Free radical initiators may be incorporated in any amounts useful to achieve the desired reaction or cure. Desirably, they are present in amounts of about 0.01% to about 10% by weight of the total composition. Combinations of the free-radical initiators are also useful.

Useful photoinitiators for formulating such compositions include, without limitation, those useful in the UV and visible light spectrums, for example, benzoin and substituted benzoins, such as benzoin ethylether, benzoin ethylether and benzoin isopropylether, benzophenone, Michler's ketone and dialkoxyacetopherones such as diethoxyacetophenone. Photoinitiators may be used in any amount effective to achieve the desired cure. Desirably, they are present in amounts of about 0.001% to about 10%, more desirably in amounts of about 0.1% to about 5% by weight of the total composition.

Useful visible light photo-initiators include, without limitation, camphorquinone peroxyester initiators, non-fluorene carboxylic acid peroxester initiators and alkyl thioxanthones, such as isopropyl thioxanthane, 7,7-dimethyl-2,3-dioxobicyclo[2.2.1]heptane-1-carboxylic acid, 7,7-dimethyl-2,3-dioxo[2.2.1]heptane-1-carboxy-2-bromoethylester, 7,7-dimethyl-2,3-dioxo[2.2.1]heptane-1-carboxymethylester and 7,7-dimethyl-2,3-dioxobicyclo[2.2.1]heptane-1-carboxylic acid chloride and combinations thereof. Diethoxyacetophenone (DEAP), diethoxyxanthone, chloro-thioxanthone, azo-bisisobutyronitile, N-methyldiethanolaminebenzophenol and combinations thereof may be used.

Heat curable compositions are among the various embodiments of the invention. Useful heat curing catalysts include, without limitation, hydrosilylation catalysts such as platinum, rhodium and their respective organohydrocarbon complexes. These heat curing catalysts may be present in amounts of about 0.01% to about 10% by weight of the total composition, and more desirably in amounts of about 0.1% to about 5% by weight of the total composition.

Moisture curing catalysts useful in compositions of the present invention include, without limitation, organometallic complexes, such as organotitinates (e.g. tetraisopropylorthotitanate, tetrabutoxyorthotitanate), metal carboxylates such as dibutyltin delaurate and dibutyltin dioctoate and combinations thereof. Moisture cure catalysts may be present in any amounts effective to achieve the intended cure. Desirable, they are incorporated in amounts of about 0.1% to about 5% by weight of the total composition.

Useful reactive silanes which can be incorporated into the inventive compositions include, without limitation, alkoxy silanes, such as tetramethoxysilane.

Useful inhibitors to enhance shelf life and prevent premature reactions may be added to various embodiments where appropriate, as well as various chelators. For example, various quinones may be employed, such as hydroquinones, benzoquinones, napthoquinones, phenanthraquinones, anthraquinones and substitutions thereof may be employed, as well as various phenols, such as 2,6-di-tert-butyl-4-methylphenol. Chelating agents such as ethylene diamine tetracetic acid (EDTA) may be employed. The inclusion and specific selection and amounts used will depend on the embodiment chosen.

In some embodiments, anaerobic compositions may be formulated from the inventive FMs, FOs or FPs. In such cases, appropriate anaerobic initiators, accelerator components and inhibitor or chelating components may be employed as described herein.

Catalysts and accelerators for anaerobically curable compositions made from the inventive compositions include any of the known catalysts and accelerators. For example sulfones such as bis(phenylsulfonemethyl)amine, N-methyl-bis-(phenylsulfonemethyl)amine, bis(p-tolylsulfonemethyl)amine, N-methyl-bis(p-tolylsulfonemethyl)amine, N-ethyl-bis(p-tolylsulfonemethyl)amine, N-ethanol-bis(p-tolylsulfonemethyl)amine, N-phenyl-ptolylsulfonemethyl-amine, N-phenyl-N-methyl-p-tolylsulfonemethyl-amine, N-phenyl-N-ethyl-p-tolylsulfonemethyl-amine, N—P-tolyl-N-methyl-p-tolylsulfonemethyl-amine, bis-(p-tolylsulfonemethypethylenediamine, tetrakis-(p-tolylsulfonemethypethylenediamine, bis-(p-tolylsulfonemethyl)hydrazine, N-(p-cholorphenyl)-p-tolylsulfonemethyl-amine, and N-(p-carboethoxyphenyl)-(p-tolylsulfonemethyl)amine may be employed. For most applications, the catalyst is used in amounts of from about 0.05 to 10.0% by weight, preferably from about 0.1 to 2% of the total composition.

The catalysts for anaerobic compositions of the present invention may be used alone in the anaerobic system or an accelerator such as orthosulfobenzimide (saccharin) may be employed in amounts of about 0.05 to 5.0% by weight of the monomer.

In anaerobic compositions, it may also be desirable to employ antioxidants, thermal stabilizers or free radical inhibitors such as teritary amines, hydroquinones, etc. in order to further prolong the shelf-like of the composition. In particular, it may be preferred to add a sterically hindered phenol, e.g. butylated hydroxytoluene (BHT), butylated hydroxyanisole (BHA), or such stabilizers as are commerically available under the tradenames Ionox 220 (Shell), Santonox R (Monsanto), Irganox 1010 and Irganox 1076 (Ciba-Geigy), etc.

Although the anaerobic compositions of the invention will cure satisfactorily under any set of anaerobic conditions, the presence of selected metals on the surface of the components to be bonded will appreciably increase the rate of curing. Suitable metals which are effective with these anaerobic compositions include iron, copper, tin, aluminum, silver and alloys thereof. The surfaces provided by the metals, alloys and their platings and which are useful in accelerating curing of these compositions will, for convenience, be grouped into the term “active metal” surfaces and be understood to include but not be limited to all of the metallic entities mentioned above. It is to be further noted that in bonding components which do not comprise these active metals (e.g. plastic, glass, non-active metal surfaces) it may be desirable to accelerate curing by pretreating these surfaces with an active metal compound which is soluble in the monomer-catalyst mixture such as ferric chloride, and cobalt, manganese, lead, copper and iron “soaps” such as cobalt-2-ethyl hexoate, cobalt butyrate, cobalt naphthenate, cobalt laurate, manganese-2-ethyl hexoate, manganese butyrate, manganese naphthenate, manganese laurate, lead-2-ethyl hexoate, lead butyrate, lead naphthenate, lead laurate, etc. and mixtures thereof. These active metal compounds may be readily applied to the surfaces, for example, by wetting the surfaces with a dilute solution of the metal compound in a volatile solvent such as trichloroethylene and then permitting the solvent to evaporate. Non-active surfaces treated in this manner can be bonded together with the sealants of the present invention as quickly as active metal surfaces.

### Preparation of Fluorinated Polymer Compositions Using Controlled Radical Polymerization Reactions

The monomeric fluorocomponents of the present invention can be polymerized using chain and step polymerizations and controlled radical polymerizations, such as by atom transfer radical polymerization (ATRP) such as by single electron transfer polymerization (SET), by stable free radical polymerization (SFRP) such as reversible deactivation by coupling, or by degenerative transfer (DT).

The fluorocompounds of the present invention, including those represented by structures I-VI, may be used in controlled radical polymerization reactions to create polymers with such properties as with increased conversion, low polydispersity, high functionality of the end products and monomodal distribution of molecular weight. These improvements are in addition to those enhanced properties discussed above which are contributed solely or largely by the fluorocompounds. Since the fluorocompounds are preferably functionalized, they will provide cites for further reaction with additional components, for further modification of the structure, for curing or a combination thereof.

ATRP, SFRP and DT are useful methods to build polymers of the present invention. The controlled or living polymerization process is one in which chain transfer and termination reactions are essentially nonexistent. These developments allow for the production of polymers that possess specific and precise quantitative functionality and chemical reactivity, with a high degree of efficiency and optimization.

Metal-catalyzed organic radical reactions and living radical polymerization (LRP), performed in nonpolar solvent systems, including mixtures of non-polar and polar systems, including reversible deactivation of the radicals are formed by disproportionation of Cu(II)X. The outer-sphere SET process has very low activation energy and thus involves fast activation and deactivation steps and negligible bimolecular termination at room temperature. FIG. 6 illustrates a proposed SET mechanism. In FIGS. 6 and 7, L is a ligane, X is a halide anion and P is polymer. For a more detailed discussion, see Percec, V. et al; “Ultrafast Syntheses of Ultrahigh Molar Mass Polymers by Metal-Catalyzed Living Radical Polymerization of Acrylates, Methacrylates, and Vinyl Chloride Mediated by SET at 25°’”, A. J. AM. Chem. Soc. 2006, 128, 14156-14165, which is incorporated herein by reference in its entirety.

One particularly useful method of controlled radical polymerization is described in US Application No. PCT/US2009/047479, published as WO2009/155303A3, and assigned to Henkel Corporation, which is incorporated by reference herein in its entirety. This application provides a method of directing the reaction mixture at a predetermined flow rate over a solid catalyst surface which is contained outside of the reaction vessel, and monitoring the temperature of the reaction vessel within a certain temperature range, adjusting the flow rate when the temperature range is outside the selected temperature range, and allowing the polymerization to proceed until a desired level of conversion is reached. This method is particularly useful in producing the fluorinated polymer compositions described herein.

Atom-transfer radical polymerization (ATRP) reactions, proceed by an innersphere electron-transfer mechanism that requires high activation energies. ATRP is considered to proceed by an innersphere electron-transfer mechanism in which a low oxidation state metal complex acts as the catalyst, mediating a fast exchange between radicals and their dormant alkyl halide species. For a more detailed description of ATRP, see K. Matyzaszewski, K. et al., “Competitive Equilibrium in Atom transfer Radical Polymerization”, Macromol. Symp 2007. 248, 60-70, which is incorporated herein in its entirety. FIG. 7 illustrates a proposed ATRP mechanism.

SET-LRP may be performed at low activation energies and thus at lower temperatures. The catalyst used regenerates itself, thus the polymerization process is living. Increasing solvent concentration of the reaction mixtures gives faster polymerization. The SET-LRP reaction starts with a SET reaction between a Cu (O) species and a halogen-containing substrate (initiator or halogen-terminated polymeric chain end). The polymerization proceeds by an outer-sphere SET mechanism in which Cu (O) species acts as electron donors, and the dominant initiator and propagating species R—X (x is a halide anion) acts as electron acceptors.

There is a continuing effort in polymer chemistry to develop new polymerization processes and new polymers. As such, Single Electron Transfer Living Radical Polymerization (hereinafter, SET-LRP) has developed and has been explored as a subset of ATRP. With the methods of the present invention, either process, or both may be practiced, yielding better results including: higher conversion rates, more efficient processes, and products with higher predictability and desirability.

There has been a continuing effort to make the controlled radical polymerization as environmentally benign and as low cost a process for the preparation of functional materials as possible. Factors such as control over the polymer molecular weight, molecular weight distribution, composition, architecture, and functionality are important considerations in the design and execution of such methods. The methods of the present invention allow for greater control over the final polymer products such that the desired chain length, polydispersity, molecular weight, and functionality are easily incorporated into the final product. Thus, the present invention overcomes the poor control over molecular weight distribution, low functionality, poor control of polymer rheology, and undesirable polydispersity. Also, because this process is so predicable, it can be easily implemented on a large scale with a high predictability and/or used to tailor the properties of the final polymer products to new degrees, and products can be designed based on their properties. Further, because there is less termination, the structure and composition of the polymer are more precise and the end product has more desirable properties and characteristics to promote a better product. Further, as very low levels of catalyst are needed to drive the reaction, purification of the final product is facilitated, and at times, unnecessary. Further, the components of the system may be optimized to provide even more precise control over the (co)polymerization of monomers.

The catalyst employed in the controlled or living polymerization processes used herein may contribute to determining the position of the atom transfer equilibrium and dynamics of exchange between dormant and active species. Thus, the catalyst employed should preferably be a good electron donor. The catalyst may be, for example: Cu(0); Cu₂S; Cu₂Te; Cu₂Se; Mn; Ni; Pt; Fe; Ru; V; CuCl; CuCl₂; CuBr; CuBr₂; and combinations thereof, and the like, as is known in the art. Similarly, other catalysts, including, for example, Au, Ag, Hg, Rh, Co, Ir, Os, Re, Mn, Cr, Mo, W, Nb, Ta, Zn, and compounds including one or more thereof may be employed with the present methods. One particularly effective catalyst is elemental copper metal, and its derivatives.

The catalyst may take one or more forms. For example, the catalyst may be in the form of a wire, mesh, screen, shavings, powder, tubing, pellet, crystals, or other solid form. The catalyst surface may be one or more of a metal, as previously disclosed or metal alloy. More particularly, the catalyst may be in the form of a copper wire, a copper mesh, a copper screen, a copper shaving, a copper powder, a copper gauze, a copper sinter, a copper filter, a copper sliver, a copper tubing, copper crystals, copper pellets, a coating of elemental copper on non-reactive materials, and combinations thereof.

The controlled polymerization methods used herein may also include the presence of a ligand, for example, a nitrogen-containing ligand which may aid in the extraction of the catalyst to the extent that the metal catalyst may be solubilized by the ligand so it is available in its higher oxidation state. Thus, the ligand may be desirable to drive the polymerization reaction to the effect that it may aid in promoting a mixture of the various components of the reaction mixture on a molecular level. A wide variety of nitrogen-containing ligands are suitable for use in the present invention. These compounds include primary, secondary, and tertiary alkyl or aromatic amines, as well as polyamines which may be linear, branched, or dendritic polyamines and polyamides. Suitable ligands for use in the present invention include ligands having one or more nitrogen, oxygen, phosphorus and/or sulfur atoms which can coordinate to the transition metal through a sigma-bond, and ligands containing multiple carbon-carbon bonds which can coordinate to the transition metal through a pi-bond. For example, suitable ligands may include tris(2-dimethylaminoethyl)amine (Me6-TREN), tris(2-aminoethyl)amine (TREN), 2,2-bipyridine (bpy), N,N,N,N,N-pentamethyldiethylenetriamine (PMDETA), and many other N-ligands.

The ligand may preferentially form a soluble complex with the redox conjugate of the transition metal, i.e. the higher oxidation state of the transition metal, forming a complex that is active in the deactivation of the growing radical chain, which may contribute to a narrow molecular weight distribution of the polymer product.

Initiators of controlled radical polymerization of the present method may initiate the free radical reaction and thusly, may be considered as contributors to the number of growing polymer chains in the reaction vessel. Suitable initiators include, for example, halogen containing compounds. Examples of initiators include chloroform, bromoform, iodoform, carbon tetrachloride, carbon tetrabromide, hexahalogenated ethane, mono-di, and tri haloacetates, acetophenones, halogenated amides, and polyamides such as nylons, halogenated urethanes and polyurethane including their block copolymers halogenated imides, acetone, and any other initiators shown to work with conventional metal catalyzed living radical polymerization including ATRP and SET-LRP. A wide variety of initiators are suitable for use in the present invention. Halogenated compounds are particularly suited for use in the invention. These initiators include compounds of the formula R—X of “R′C(═O)OR” where X is a halogen and R is C1-C6 alkyl. For example, the initiator may include: diethyl meso-2,5-dibromoadipate; dimethyl 2,6-dibromoheptanedioate, ethylene glycol bis(2-bromopropionate); ethylene glycol mono-2-bromopropionate; trimethylolpropane tris(2-bromopropionate); pentaerythritol tetrakis(2-bromopropionate); 2,2-dichloacetophenone; methyl 2-bromopropionate; methyl 2-chloropropionate; N-chloro-2-pyrrolidinone; N-bromosuccinimide; polyethylene glycol bis(2-bromopropionate); polyethylene glycol mono(2-bromopropionate); 2-bromopropionitrile; dibromochloromethane; 2,2-dibromo-2-cyanoacetamide; α,α′-dibromo-ortho-xylene; α,α′-dibromo-meta-xylene; α,α′-dibromo-para-xylene; α,α′-dichloro-para-xylene; 2-bromopropionic acid; methyl trichloroacetate; para-toluenesulfonyl chloride; biphenyl-4,4′-disulfonyl chloride; diphenylether-4,4′-disulfonylchloride bromoform; iodoform carbon tetrachloride; and combinations thereof. In some embodiments, the initiator may be an alkyl, sulfonyl, or nitrogen halide. The nitrogen halide can be also halogenated nylon, peptide, or protein. Alternatively, a polymer containing active halide groups, for example, poly(vinyl)chloride), the chloromethyl group or polychrolomethylsytrene) of the polymers and copolymers can also be used as initiators.

Once the polymerization is complete, the method may include further reacting the resultant polymer to form at least one functional end group onto the polymer. The functionality of the intermediate product creates a multi-use end product that may be converted into one or more final products. The final products may then be implemented into various commercial products or procedures, as may be desired. In order to quench the reaction and terminate the process, strong nucloephiles may be added to the reaction mixture. Such nucleophiles include, for example: thiolate, amine, azide, carboxylate, alkoxide, and sodium carboxylate. One or a combination of nucleophiles may be used as may be desired in order to terminate the reaction while maintaining chain stability and integrity. Creating functional ends on the polymer may be done, for example, by performing either an end-capping reaction or a substitution reaction.

To functionalize the final product polymer by an end-capping reaction, the required steps may be done in situ in the reaction vessel at the end of the initial reaction, prior to work-up. To perform an end-capping functionalization of at least one polymer end, the steps include: providing a final polymer product; adding a capping agent to the vessel; quenching the reaction; and purifying a capped polymer product.

The capping agent may include one or a combination of compounds, as may be desired to cap the end group of the final product with a desired functional end group while maintaining chain stability and integrity. For example the capping agent may include: 2 allyl alkyl ethanol, allyl alcohol, allyl glycidyl ether, 1-6 heptadiene, cyclooctyl diene, norbornadiene, and other olefins with a known tendency to not form homopolymers under SET-LRP conditions.

The final products of the methods of the present invention include, for example, homopolymers and/or (co)polymers, which may be block, random, statistical periodic, gradient star, graft, comb, (hyper)branched or dendritic polymers. The “(co)” parenthetical prefix in conventional terminology is an alternative, viz., “(co)polymer means a copolymer or polymer, including homopolymer. Similarly “(hyper)” as used herein, refers to a comparatively high degree of dendritic-like branching along the co-polymer backbone as compared to a low degree of branching.

The present invention may be used to prepare periodic or alternating copolymers. The methods of the present invention may be particularly useful for producing alternating copolymers where one of the monomers has one or two bulky substituents, from which homopolymers may be difficult to prepare, due to steric considerations. Copolymerization of monomers with donor and acceptor properties results in the formation of products with predominantly alternating monomer structure.

So-called “alternating” copolymers can be produced using the methods of the present invention. “Alternating” copolymers are prepared by copolymerization of one or more monomers having electron-donor properties with one or more monomers having electron acceptor type properties (acrylates, methacrylates, unsaturated nitriles, unsaturated ketones, etc.). The present random or alternating copolymer can also serve as a block in any of the present block, star, graft, comb or hyperbranched copolymers.

The end product may be characterized by one or more features, including: molecular weight, polydispersion, monomodal distribution of molecular weights, etc. One or more of the methods of the present invention may yield a polymer product having a molecular weight of 2,000 to 20,000,000 g/mol. Also, the polymer product has a monomodal distribution of polymer molecular weights. Further, the polymer product may also have a polydispersity from about 1.01 to about 2. In certain embodiments, the polymer produced by the process described herein has a number average molecular weight of at least about 500. In yet other embodiments the polymer has a number average molecular weight of at least 1,000,000.

Any of the fluorinated compounds disclosed may be used with any of the other disclosed reactive components to provide various embodiments. Various fluorinated monomers may be added alone or as blends to polymerizable compositions including the various additional monomers, initiators, catalysts, diluents, stabilizers, fillers, plasticizers and other components described herein. The various combinations of the herein described components are intended to be included within the various embodiments of the invention.

### EXAMPLES

### Example 1

### 2,2,3,3,4,4,5,5,-Octafluoropentoxy trimethoxy silane Synthesis

[chemical structure]

| | tetra- | | | | | |
|---|---|---|---|---|---|---|
| | methoxy- | | | Reaction | | |
| Reagents | silane | CH3ONa | OFP | Temp. | Yield | |
| Amount | 548 g | 2.14 g | 557 g | 130° C. | 650 g | 77% |
| Mole | 3.6 mol | 0.04 mol | 2.4 mol | | | |

### Experiment Section

Mix tetramethoxysilane (548 g, 3.6 mol) and CH₃ONa (2.14 g, 40 mmol)
Heat the mixture at 130° C.
Add 2,2,3,3,4,4,5,5-octafluoro-1-pentanol (557 g, 2.4 mol) dropwise
Remove distillate at the boiling point of methanol until the weight of distillate approximately equals to the theoretical amount of methanol (˜4 hrs).
Cool down the reaction mixture
Perform distillation under reduced pressure
Collect the clear fraction at 60° C./40 mmHg.
Total product (Compound II) 650 g, 77% yield.

### Example 2

### Synthesis of C18-octafluoropentyl-diethyl orthosilicate (Compound Va)

[chemical structure]

Operating procedure:

| Substrate | Quant | moles | Equivalents |
|---|---|---|---|
| tetraethoxysilane | 124.8 g | 0.6 mol | 1.0 |
| octafluoropentanol | 139.2 g | 0.6 mol | 1.0 |
| stearyl alcohol | 162 g | 0.6 mol | 1.0 |
| sodium methoxide | 1.3 g | 0.024 mol | 0.04 |

Into a dried 1-L three-neck flask with a magnetic stirring bar in oil bath at room temperature, was subsequently added stearyl alcohol (162 g), tetraethoxysilane (124.8 g), octafluoropentanol (139.2 g), sodium methoxide (1.3 g). After addition was finished, the round bottom flask was connected to a distilling apparatus. The oil temperature was raised to 170° C., and meanwhile ethanol was removed by distillation in the duration of the reaction. After 30 hours, the temperature of oil bath was lowered to 70° C. Then the product was stirred at 70° C. under vacuum (5 mm Hg) for 12 h. Total amount 360 g product was obtained. The detection by ¹H NMR and ¹⁹F NMR showed that the yield of the target product (Compound Va) was approximate 90%. The other 10% are by-products and are characterized as compound A or B, C.

[chemical structure]

### Example 3

### Preparation of mono-OFP urethanated 1,3-bis(2-isocyanatopropan-2-yl)benzene (Compound V)

[chemical structure]

### Reaction Procedure:

Under N₂ atmosphere, OFP (139 ml, 1.0 mol), 1,3-bis(2-isocyanatopropan-2-yl)benzene (1.0 mol) and hexane (700 ml) was mixed in a 2 L three-necked flask at room temperature. After the reaction system was cooled by an ice-salt bath to −5° C., dibutyltin dilaurate (6.3 g, 10.0 mmol) was added drop wise. The reaction system temperature was maintained ≦0° C. during dibutyltin dilaurate addition. Then, the mixture was allowed to warm to room temperature slowly. There was white precipitation which appeared when the mixture was stirred for 1 h. After 10 h, the reaction was completed. The white precipitation was identified as containing desired product, Compound 3. The desired product ((Compound V) was obtained by recrystallization (358 g, 82.7% yield).

### Example 4

### Preparation of mono-OFP urethane, mono-isopropenyl benzene (Compound III)

[chemical structure]

Under N2 atmosphere, 2,2,3,3,4,4,5,5-octafluoro-1-pentanol (139 ml, 1 mol), 1-(2-isocyanatopropan-2-yl)-3-(prop-1-en-2-yl)benzene (237 ml, 1.2 mol) and hexane (700 ml) were charged to a 2 L three-neck flask while stirring. The reaction mixture was then cooled to −5° C. by an ice-salt bath. Dibutyltin dilaurate (6.3 g, 10 mmol) was added to reaction mixture dropwise. The reaction system temperature was maintained below 0° C. during dibutyltin dilaurate addition. Then the mixture temperature was allowed up to room temperature gradually and was stirred at this temperature for 30 h. The proportion of desired product (Compound III) to reactant 2,2,3,3,4,4,5,5-octafluoro-1-pentanol is 100:1. After the removal of volatile solvents under vacuum, the crude desired product was further purified by silica gel flash column chromatography (PE to PE:EA 20:1). The yield was (500 g, 79.8%).

### Example 5

This example demonstrates the use of controlled radical polymerization to form a novel telechelic polymer of butyl acrylate and octafluoropentyl (meth)acrylate. 2-hydroxyethylacrylate was additionally incorporated to provide hydroxyl functionality.

[chemical structure]

In a pressure tube, butyl acrylate (4.5 g), octafluoropentyl acrylate (1.5 g), diethyl 2,5-dibromoadipate (0.108 g), Me6Tren (0.01035 g) and CuBr2 (2.2 mg) were added and then degassed for 15 min. using dry nitrogen gas, then 3.43 mg copper powder was added under nitrogen atmosphere. The pressure tube was sealed, and the reaction allowed to proceed 14 hours at 70-75° C. Then 0.69 grams of 2-hydroxyethyl acrylate was added and the reaction was allowed to proceed for another 4 hours at 70-75° C. The reaction was then quenched by exposure to oxygen and then passed through an alumina column. Unreacted monomer was removed by precipitating in methanol. The NMR characterization indicated the polymer contained 74.8% wt. butyl acrylate, 24.0% wt. octafluoropentyl acrylate and 1.2% wt. 2-hydroxyethyl acrylate. GPC showed that MN=19,400 and PDI=1.24.

### Example 6

The same components of Example V were used except that the pendant hydroxyl group from 2-hydroxyethyl acrylate end-cap was further reacted with isocyanatopropyltrimethoxysilane to provide moisture curing endcapped trimethoxysilane functionality.

[chemical structure]

The fluoro-containing polymer with pendant hydroxyl groups from Example V can be capped with isocyanatopropyltrimethoxysilane in situ prior to moisture cure formulation. In a typical experiment for a polymer with hydroxyl number of 23.9 mg KOH/g, 250 gram of polymer, 21.86 gram of isocyanatopropyltrimethoxysilane and 271 mg of dibutyltin dilaurate (catalyst) are added to a Ross mixer. After 5 min mixing with rpm=5, apply vacuum slowly until full vacuum can be applied without resin going up. Then raise temperature to 90° C. and keep mixing at 18 rpm for 2 hours. The desired product will contain trimethoxysilane group which can be used for moisture cure formulation.

### Example 7

The same components of Example IV were used except the 2-hydroxyethylacrylate endcap is further reacted with acrylic acid or acryloly chloride to provide UV curing end groups.

In a typical experiment for a polymer with hydroxyl number of 23.9 mg KOH/g, 250 gram of polymer, 9.59 gram of acrylic acid (25% mol excess over —OH) and 521 mg of MEHQ (inhibitor, 200 ppm) are added to a Ross mixer. After 5 min mixing with rpm=5, apply vacuum slowly until full vacuum (20 mmHg) can be applied without resin going up. Then raise temperature to 110° C. and keep mixing at 18 rpm under vacuum for 2 hours. The desired product will contain acrylate group which can be used for UV cure formulation.

### Example 8

This example demonstrates the use of the inventive functionalized perfluoromonomers as adhesion promoters for perfluoropolymer films, such as Nafion® films, commercially available from DuPont, Wilmington, Del. This example uses 2,2,3,3,4,4,5,5-octafluoropentoxytrialkoxysilane as the functionalized perfluoromonomer in a solvent-based primer solution to improve Van der Waals interaction with Nafion (perfluoropolymer) film. In addition to the functionalized perfluoromonomer, other non-fluorinated adhesion promoters, including vinyltrimethoxysilane (VTMS), allytrimethoxysilane (ATMS) and 7-octenyltrimethoxysilane (OTMS) were also incorporated in the primer solution to covalently bond to hydrosilyzation-cured adhesive gasketing material.

| | Composition | Weight |
|---|---|---|
| 2,2,3,3,4,4,5,5,- | 30-60 wt. % | 50 wt. % |
| Octafluoropentoxy | | |
| Ti(IV) butoxide (catalyst) | 5-10 wt. % | |
| Other functional trimethoxy | 5-20 wt. % | |
| silane | | |
| Heptane | 30-60 wt. % | |

Priming Condition

| | | |
|---|---|---|
| Temperature | Room temperature | |
| Relative Humidity | 40-50% | 50% |
| Reaction Time | 30-90 min. | |

In a glass bottle was added 63.13 g Dow Corning 532260 (15-40% VTMS, 1-5% Ti(IV) butoxide, >60% light aliphatic petroleum solvent naptha and 63.17 g 2,2,3,3,4,4,5,5-octafluoropentoxytrimethoxysilane. The bottle was capped, shaken well and was then applied as a surface primer to a Nafion perfluoropolymer film surface and dried for about 30 minutes at room temperature (RT) and about 50% Relative Humidity (RH). A heat-curable hydrocarbon gasketing product was then applied over the primed Nafion surface, and cured at 120° C. for 45 Min. 100% cohesive failure was observed on Nafion surface by peeling test.

### Example 9

This example demonstrates another primer/surface modifier composition of the present invention. In a glass bottle was added 25.7708 g heptane (anhydrous), 3.4823 g Ti(IV)butoxide, 5.5816 g ATMS, and 34.798 g 2,2,3,3,4,4,5,5-octafluoropentoxytimethoxysilane. The bottle was capped and shaken well and ready for use. The primer solution was then applied to a Nafion perfluoropolymer film and allowed to set for about 30 minutes at RT, and about 50% RH. A heat-curable hydrocarbon gasketing product was then applied over the primed Nafion surface, and cured at 120° C. for 45 min. 100% cohesive failure was observed on Nafion surface by peeling test.

What is claimed:

1. A composition comprising a compound of the structure I: [chemical structure]
wherein,
R and R¹ may be the same or different and each may be selected from H, alkyl C₁₋₁₈ substituted or nonsubstituted;
R² may be selected from siloxy, alkylsiloxyether, [chemical structure]
R³ may be selected from aromatic, aliphatic or cycloaliphatic;
R⁴ may be selected from H, alkyl C₁₋₁₈ substituted or unsubstituted, [chemical structure]
R⁵ may be selected from an aliphatic or aromatic group which may be substituted or unsubstituted and which may include one or more unsaturated groups;
R⁶ may be selected from a substituted quarternary amine or a metal cation (M+);
R⁷ may be selected from H, alkyl C₁₋₁₈ substituted or unsubstituted, NR³R⁴, OR⁸ or F;
R⁸ may be selected from alkyl C₁₋₂₀ substituted or unsubstituted;
n is 1-4, and [symbol] indicates the point of attachment to the structure.

2. The composition of claim 1, wherein Compound I is grafted onto a backbone.

3. The composition of claim 1, wherein Compound I includes a surfactant moiety.

4. The composition of claim 1, wherein the compound is: [chemical structure]
wherein each occurrence of R⁹ may be the same or different and may be selected from the group consisting of H, alkyl C₁₋₂₀ and combinations thereof; or [chemical structure]
wherein R³ may be selected from aromatic, aliphatic or cycloaliphatic; or

[chemical structure]

5. A reaction product of:
i) A compound of the structure I: [chemical structure]
wherein,
R and R¹ may be the same or different and each may be selected from H, alkyl C₁₋₁₈ substituted or nonsubstituted;
R² may be selected from siloxy, (meth)acryloxy, vinyl ether, epoxy ether, alkyl ether, [chemical structure]
R³ may be selected from aromatic, aliphatic or cycloaliphatic;
R⁴ may be selected from H, alkyl C₁₋₁₈ substituted or unsubstituted, [chemical structure]
R⁵ may be selected from an aliphatic or aromatic group which may be substituted or unsubstituted and which may include one or more unsaturated groups;
R⁶ may be selected from a substituted quarternary amine or a metal cation (M+);
R⁷ may be selected from H, alkyl C₁₋₁₈ substituted or unsubstituted, NR³R⁴, OR⁸ or F;
R⁸ may be selected from alkyl C₁₋₂₀ substituted or unsubstituted; and
n is 1-4; [symbol] indicates the point of attachment to the structure; and
ii) a free radical initiator.

6. A method of forming a fluorinated moisture curing silane comprising:
i) mixing an organo silane compound and an alkali or alkaline earth metal oxide in a reaction vessel under heat; and
ii) further combining the resultant mixture with a fluorinated alkanol and permitting the reaction to proceed to form a moisture curing fluorinated silane.

7. A method of forming a fluorinated curable composition through controlled polymerization comprising:
i.) combining the composition of claim 1 with a free radical initiator, a ligand capable of coordinating with a metal catalyst and a metal catalyst in a reaction vessel;
ii) permitting the reaction to proceed at a suitable temperature and for a suitable time until the desired degree of polymerization is reached, wherein a fluorinated polymerizable compound is produced.

8. An adhesive, sealant or coating composition comprising:
(i) one or more compounds of claim 1;
(ii) one or more reactive components selected from the group consisting of monomers, polymers, oligomers, reactive diluents and combinations thereof; and
(iii) a cure system.

9. A composition comprising a compound which includes in its structure the segment: [chemical structure]
wherein R₁₀ and R₁₁ may be the same or different and may be independently selected from H, C₁₋₁₀ substituted or unsubstituted hydrocarbon; R¹² may be selected from hydrophobic monomers, hydrophilic monomers, aromatic monomers, aliphatic monomers and combinations thereof; and
wherein X represents mole % from 0.001-100 and y represents mole % from 0-99.999%.

10. The composition of claim 9, wherein the segment includes at least one reactive terminal group.

11. The composition of claim 9, wherein the segment is part of a polymer or oligomer which is predominantly hydrophobic or predominantly hydrophilic.

12. A controlled polymerization process comprising:
(i) providing a compound of the structure: [chemical structure]
wherein,
R and R¹ may be the same or different and each may be selected from H, alkyl C₁₋₁₈ substituted or nonsubstituted;
R² may be selected from siloxy, (meth)acryloxy, vinyl ether, epoxy ether, alkyl ether, cyanoacrylate, cyanoacetate. [chemical structure]
R³ may be selected from aromatic, aliphatic or cycloaliphatic;
R⁴ may be selected from H, alkyl C₁₋₁₈ substituted or unsubstituted, [chemical structure]
R⁵ may be selected from an aliphatic or aromatic group which may be substituted or unsubstituted and which may include one or more unsaturated groups;
R⁶ may be selected from a substituted quarternary amine or a metal cation (M+);
R⁷ may be selected from H, alkyl C₁₋₁₈ substituted or unsubstituted, NR³R⁴, OR⁸ or F;
R⁸ may be selected from alkyl C₁₋₂₀ substituted or unsubstituted; R⁹ may be selected from H, alkyl C₁₋₁₈ substituted or unsubstituted, NR³R⁴, OR⁵ or F; n is 1-4; and [symbol] indicates the point of attachment to the structure.
(ii) combining the compound with an organometallic compound or a hydride of Group IV-VIII transition metals to form a reaction mixture; or combining the compound with a free radical initiator and a chain transfer agent to form a reaction mixture; and
(iii) reacting a mixture at a sufficient temperature and time to form a polymer.

13. The process of claim 12, wherein one or more of the following groups is present in the resulting polymerization product: methacryloxy, hydroxy, epoxy, alkoxy, amino, isocyanate, vinyl, allyl, siloxy, halo and carbamate.

14. The process of claim 12, further comprising the step of functionalizing the resultant polymerized product.

15. The process of claim 12 further incorporating an additional reactive component.
